(19) United States
(12) Patent Application Publication
(10) Pub. No.: US 2002/0012980 A1
(43) Pub. Date: Jan. 31, 2002

(54) Method for simultaneous saccharification and fermentation of spent cellulose sausage casings

(75) Inventor: Hassan K. Sreenath, Madison, WI (US)

Correspondence Address: MICHAEL BEST & FRIEDRICH, LLP, ONE SOUTH PINCKNEY STREET, P O BOX 1806, MADISON, WI, 53701

(73) Assignee: Wisconsin Alumni Research Foundation, Madison, WI (US)

(21) Appl. No.: 09/915,028

(22) Filed: Jul. 25, 2001

Related U.S. Application Data
(60) Provisional application No. 60/220,789, filed on Jul. 25, 2000.

(51) Int. Cl. C12P 21/04; C12P 7/56; C12P 1/04

(52) U.S. Cl. 435/139; 435/170; 435/855; 435/853; 435/857

(57) ABSTRACT

Disclosed are methods for bioconversion of cellulose in spent sausage casings to a useful end-product. The methods involve treating the cellulose with a cellulase to yield glucose, which in turn is converted to lactic acid, ethanol, enzymes or feed grade protein by a microorganism.

### CROSS REFERENCE TO RELATED APPLICATIONS

This application claims priority to U.S. Provisional Application No. 60/220,789, filed Jul. 25, 2000.

### BACKGROUND OF THE INVENTION

Cellulose sausage casings are used extensively in the manufacture of sausages. In large-scale operations, cellulose sausage casings are preferred over collagen casings derived from animal intestines because cellulose casings are stronger, have a more even consistency, and are less expensive than collagen casings. The cellulose casings are used to make skinless frankfurters, which are made by packing the cellulose casing with meat product, twisting the ends shut to seal, and cooking the meat. After cooking, the casing is stripped off and discarded.

The annual production of cellulose sausage casings in the United States exceeds 14 million kg dry weight. During cooking, the casings absorb water, causing an increase in weight and volume. Disposal of spent sausage casings is problematic, and the high cost of landfill disposal of the spent casings is a serious economic concern for the sausage industry. There are also environmental costs associated with the disposal of spent casings. Available landfill space is diminishing. The spent casings are susceptible to spoilage, resulting in the possible production of toxins or foul odors. Disposal of cellulose casings in landfills raises concerns about potential contamination of the water table with organic waste. At least one state has called upon the sausage industry to find alternatives to landfills for disposing of spent cellulose sausage casings.

What is needed in the art is an economical, environmentally friendly means of disposing of or utilizing spent cellulose sausage casings.

### BRIEF SUMMARY OF THE INVENTION

In one aspect, the present invention provides a method for converting spent cellulose sausage casings comprising treating the sausage casings with cellulase and at least one lactic acid-producing bacteria under suitable conditions and for a period of time sufficient to allow conversion of at least a portion of the cellulose to lactic acid.

In another embodiment, the present invention provides a method of converting cellulose in cellulose sausage casings to ethanol comprising treating spent cellulose sausage casings with cellulase and an ethanol producing microorganism under suitable conditions and for a period of time sufficient to allow conversion of at least a portion of the cellulose to ethanol.

In another aspect, the present invention provides a method for converting spent cellulose sausage casings comprising growing cellulolytic fungi on casings in a solid substrate cultivation (SSC) to produce cellulase; saccharifying cellulose in spent cellulose sausage casings by combining the enzymes produced in the solid substrate cultivation step with additional spent cellulose sausage casings and an organism capable of converting glucose to lactic acid or ethanol. Optionally, the ethanol or lactic acid may be removed continuously or periodically from the fermentation mixture. Preferably, the saccharification and fermentation steps are conducted simultaneously.

The present invention also provides a method of producing an enzyme from the solid substrate cultivation of a cellulolytic fungi comprising inoculating spent cellulose sausage casings with a cellulolytic fungi; and incubating the inoculated casings under suitable conditions and for a period of time sufficient to allow the fungi to produce an enzyme selected from the group consisting of cellulase, xylanase, hemicellulase, and pectinase.

In another aspect, the present invention provides a method of producing feed grade protein from spent cellulose sausage casings with a cellulolytic fungus. The solid substrate cultivation of a cellulolytic fungus comprises spent casings as solid substrate inoculated with the fungus incubated under suitable conditions and for a period of time sufficient to allow the fungus to hydrolyze at least a portion of the cellulose to glucose and to convert at least a portion of the glucose to enzymes or other proteins.

It is an advantage that the method of the present inventions permits conversion of the cellulose in sausage casings without requiring that the casings be washed to remove contaminating nitrates, nitrites, and salt, which tend to inhibit microbial growth.

The method of the invention produces end products that are useful in diverse industries such as food, plastic, or fuel industries while, at the same time, reduces economic and environmental costs associated with disposal of spent cellulose sausage casings.

### BRIEF DESCRIPTION OF THE DRAWING

FIG. 1 shows the percent conversion of cellulose during ethanol fermentation of spent cellulose sausage casings incubated with cellulase, cellulase plus Kluveromyces marxianus yeast, or without cellulase and yeast.

### DETAILED DESCRIPTION OF THE INVENTION

The present invention provides methods for the bioconversion of cellulose in spent sausage casings to lactic acid, ethanol, enzymes, or feed proteins by the simultaneous saccharification of cellulose and fermentation of glucose. Bioconversion of cellulose not only yields useful end products, but reduces or eliminates the cost of landfilling.

Cellulose is a plant polysaccharide comprised of a polymer of glucose units linked by β-1,4 glucosidic bonds. Using the invention described herein, the cellulose in spent sausage casings is converted to glucose by placing the casings in a suitable medium and contacting with the enzyme cellulase, which catalyzes the hydrolysis of cellulose to form a combination of smaller oligosaccharides, disaccharides (cellobiose), and glucose. The cellulase may be provided in the form of a purified or partially purified enzyme. Preferably, the cellulase may be provided most economically by on-site cellulase production. Cellulase production may be achieved by solid substrate cultivation of any suitable fungal species capable of producing a cellulolytic enzyme on cellulose from spent sausage casings. Following cultivation of the fungal species, the solid substrate is mixed with spent cellulose sausage casings and the mixture is inoculated with a suitable microorganism, such as a bacteria, yeast or fungi, capable of converting free sugars to a useful end-product. For example, free glucose produced by the cellulase-catalyzed hydrolysis of cellulose may be converted to lactic acid by lactic acid-producing microorganisms or to ethanol by microorganisms capable of fermenting glucose to ethanol. Lactic acid thus produced may be used as a food additive or in the manufacture of plastics. Ethanol produced by the method of the invention may be used as fuel or as an industrial feedstock.

Spent casings contain large concentrations of salt, nitrate, and nitrite, which can be inhibitory toward the growth of and fermentation by microorganisms. Although contaminants could be removed from the cellulosic material by washing prior to SSF, such a step in the process of casing disposal or conversion would add considerably to the cost of disposal or conversion, lower the economic feasibility of the process, and create waste water, which would also have to be disposed. Surprisingly, the microorganisms tested were shown to exhibit very high conversion of cellulose to glucose and fermentation to ethanol or lactic acid on unwashed, spent sausage casings, despite the presence of high concentrations of microbial inhibitors.

Preferably, the cellulolytic fungi used in the method of the invention are fungi approved by the food and drug administration and are generally recognized as safe (GRAS). Examples of GRAS status cellulolytic fungi suitable for use in the method of the present invention include, but are not limited to, Trichoderma reesei, Rhizopus oryzae, and Aspergillus niger. The cellulolytic fungi grown on spent cellulose sausage casings by SSC will hydrolyze the cellulose, and use the sugar from the hydrolyzed cellulose and nitrogen from the nitrogen in residual meat juices for growth and the production of cellulase and other proteins. Optionally, the solid substrate may be supplemented with additional nutrients, such as nutrient nitrogen.

By “simultaneous saccharification and fermentation” it is meant that the cellulose sausage casings are treated with: (1) exogenous cellulase or cellulase produced by a solid substrate cultivation on spent cellulose sausage casings by a cellulase-producing microorganism and (2) a microorganism capable of converting glucose to lactic acid, ethanol, enzymes or feed proteins at essentially the same time, in the same medium, and under the same conditions.

By “cellulose sausage casings” it is meant fibrous or transparent cellulose material used in the preparation of sausages, frankfurters, and the like. The casings may be made from viscose cellulose.

As demonstrated in the Examples, efficient conversion of cellulose to lactic acid was achieved in a medium suitable for simultaneous saccharification and fermentation (SSF) using spent cellulose sausage casings, commercial cellulase, and an inoculum of the Lactobacillus delbrueckii or Lactobacillus plantarum. The spent sausage casings may be shredded and possibly blended with other cellulosic fiber to improve aeration for consistent fungal growth during solid substrate cultivation.

The fermentation medium for lactic acid production of the Examples included CaCO₃ as a buffer to maintain the pH of the fermentation within a suitable range from about 5.5 to about 6.0.

The activity of the enzyme cellulase increases with increasing temperature up to a maximum of from about 50-80° C. Therefore, the simultaneous saccharification fermentation reaction is preferably conducted at a temperature at the high end of the range of temperatures for optimal growth of the lactic acid-producing microorganism used in the fermentation. In the Examples, fermentations were conducted at 37° C. It is expected that more efficient conversion of glucose to lactic acid could be obtained using an incubation temperature in the range of from about 37° C. to about 41° C. for L. plantarum, or about 40° C. to about 45° C. for L. delbrueckii.

Cellobiose, one of the products of the hydrolysis of cellulose by cellulase, is inhibitory for cellulase activity. L. delbrueckii and L. plantarum are able to utilize cellobiose, thereby preventing the inhibition of cellulase activity caused by high concentrations of cellobiose. L. plantarum is able to metabolize cellobiose more rapidly than L. delbrueckii. If one wished to employ a lactic acid-producing microorganism that was unable to use cellobiose, simultaneous saccharification fermentation could be enhanced by supplementing the medium with exogenous β1,4 glucosidase.

It is expected that other lactic acid-producing microorganisms in addition to L. delbrueckii or L. plantarum may be used in the practice of the present invention. Other suitable microorganisms include bacteria, yeast, or fungi capable of fermenting glucose to produce lactic acid. It is envisioned that improved strains of microorganisms having advantageous properties or improved qualities may be selected or genetically engineered for use in the practice of the present invention. Examples of such improved useful qualities include, but are not limited to, enhanced lactic acid production, increased expression of β-glucosidase, increased acid tolerance, and increased optimal growth temperature.

The method may be conducted in batch, semi-continuous, or continuous mode. Preferably, the method allows recovery and reuse of the cellulase, the lactic acid-producing microorganisms, or both. It is envisioned recycling of the cellulase, the lactic acid-producing microorganisms, or both may be facilitated by immobilization or microencapsulation of the catalyst or biocatalyst by any suitable means.

The cellulase activity needed to provide efficient saccharification will depend on a variety of factors, including the initial concentration of cellulose, reaction conditions, desired time of incubation, the stability of the enzyme under the incubation conditions, and the extent of saccharification desired. In the Examples below, the activity of cellulase used was about 25 to about 50 filter paper cellulase units (FPU) in 50-ml reaction volumes containing 10% (w/v) cellulose. Preferably, the cellulase is added in an amount sufficient to provide activity that will convert most of the cellulose to disaccharides or monosaccharides.

Any suitable cellulase preparation, including cellulase preparations that are available commercially, may be used for saccharification of the cellulose casings. Examples of good commercial cellulase preparations include, without limitation, Mutifect B cellulase (Genencore); Spezyme® (Genencore); Celluclast 1.5 L-cellulase (NOVO); Novozyme 342 (NOVO); Superace AARL-Cellulase; and NCE-L-600 AARL-cellulase. Preferably, the cellulase may be provided by the solid substrate cultivation of a cellulolytic fungus using spent cellulose sausage casings as the substrate, as described below in the examples. It is envisioned that the cellulase from SSC may be provided as part of the SSC, or it may be partially purified from the SSC.

The lactic acid produced by the simultaneous saccharification and fermentation reaction may be isolated as lactic acid from the medium, or may be recovered by precipitation from the medium in a salt form, such as calcium lactate. The lactic acid may be in the L- or D-form, or a combination thereof. It is noteworthy that when L. delbrueckii is used as the biocatalyst in the method of the invention, L-lactic acid, the form used in the pharmaceutical and food industries, predominates (95%). The insoluble salt form could be formed by adding additional calcium carbonate. It is envisioned that the calcium carbonate could be provided as cellulosic fines from wood pulp, which contains large amounts of calcium carbonate. The cellulosic fines would not only provide a rich source of calcium carbonate, but would also provide cellulose, which could be saccharified and fermented to produce lactic acid.

In the Examples below, Kluveromyces marxianus was used to ferment glucose to produce ethanol in simultaneous saccharification fermentation reaction. However, any suitable organism capable of converting glucose to ethanol may be used in the practice of the invention. K. marxianus was used because it is able to ferment glucose at relatively high temperatures (45° C.). Other Kluveromyces species are also suitable for use in the method of the invention. As discussed above, cellulase activity is greater at higher temperatures. Therefore, the organism used in fermentation is preferably one that is able to grow and ferment glucose at the relatively high temperatures at which cellulase activity is maximal. However, it is specifically envisioned that microorganisms such as Saccharomyces cerevisae, which has a lower growth and fermentation temperature, could also be used in the practice of the present invention. A yeast that has been genetically engineered or selected on the basis of its ability to grow and ferment glucose to produce ethanol at higher temperatures would be useful in the practice of the present invention.

In the Examples, the sausage casings used in SSC or in SSF were steam-sterilized prior to inoculation with the cellulolytic fungus or glucose-fermenting microorganism. It is envisioned that the method of the invention may preferably be practiced using unsterilized sausage casings or other fermentation components provides that the organism used in SSC or SSF is able to outgrow contaminating microorganisms. For example, Aspergillus niger has been found to grow well under non-sterile conditions.

The following nonlimiting Examples are intended to be purely illustrative.

Preparation of Lactobacillus Culture

Lactobacillus delbrueckii or Lactobacillus plantarum was grown as a lawn on Lactobacilli MRS agar medium for 48 h at 32° C. To prepare an inoculum for a 50-ml simultaneous saccharification fermentation reaction, the bacterial lawn was transferred into 5 ml of a 10× nutrient supplement medium to give an inoculum with cells at a concentration of 6 grams (dry weight)/L. The 10× medium contains yeast extract (5 g/L), Bacto peptone (10 g/L), sodium citrate (2 g/L), sodium acetate (5 g/L), K₂HPO₄ (2 g/L), MgSO₄ .7 H₂O (0.58 g/L), MnSO₄.7 H₂O (0.12 g/L), FeSO₄ .7 H₂O (0.05 g/L), and Tween 80 (1 g/L). A 5-ml inoculum thus prepared is sufficient to provide an initial cell concentration of about 0.6 g cells (dry weight)/L in a 50-ml SSF reaction.

Lactic Acid Analysis

The simultaneous saccharification fermentation medium was sampled periodically to monitor sugar concentrations and lactic acid production. The samples were cleared by centrifugation. A 100 μl aliquot of the supernatant was diluted 1:10 with water. Sugars and by-products were analyzed by high performance liquid chromatography (HPLC) using ION 300 column (30.0×7.8 mm) using a mobile phase of 0.005 N sulfuric acid at flow rate of 0.4 ml/min at 65° C.

Simultaneous Saccharification and Fermentation of Dry Casings to Produce Lactic Acid

Initial simultaneous saccharification and fermentation (SSF) reactions were conducted in 50 ml reaction volumes in 125-ml flasks. The cellulose sausage casings were steam-sterilized, whereas all other fermentation components were added aseptically. The SSF reaction contained shredded transparent or fibrous cellulose sausage casings (5 g), CaCO₃ (2.5 to 5 g), 1 ml cellulase (2% w/v, 25 filter paper units (FPU)) (Genencore), a 5-ml culture of Lactobacillus delbrueckii or Lactobacillus plantarum containing about six grams of cells (dry weight) per liter in 10×nutrient supplement medium, and sufficient H₂O to bring to a volume of 50 ml. The culture had an initial pH of about 5.5 to 6.0. The flasks were incubated at 37° C. with shaking at 180 rpm.

High productivity of lactic acid was consistently obtained in 50-ml reaction volumes using the following conditions: sausage casings (100 g/L); 44 ml H₂O; 1.0 ml commercial cellulase (2% w/v; 25 FPU/ml) (Genencore); 2.5 g CaCO₃; 5-ml of a Lactobacillus inoculum sufficient to give an initial cell concentration of 0.6 g/L cells (dry weight) in 10× nutrient supplement.

Efficient conversion of cellulose to lactic acid was obtained in fermentations conducted in the presence of either L. plantarum or L. delbrueckii. With L. plantarum, 100 g/L lactic acid was obtained from 100 g/L of fibrous or transparent cellulose casings; with L. delbrueckii, 100 grams of transparent cellulose yielded 94 grams of lactic acid. With starting cellulose concentrations of 100 g/L, lactic acid production averaged from 2.3 to 2.44 g/L/h over a 41 hour fermentation.

Simultaneous Saccharification and Fermentation of Spent Casings to Produce Lactic Acid

To evaluate whether efficient conversion of cellulose to lactic acid could be obtained using spent casings, simultaneous saccharification and fermentation experiments were conducted essentially as described in the previous section using spent cellulose casings obtained from the Muscle Biology Laboratory, University of Wisconsin (Madison, Wis.). The spent casings contained 20-30% water, as well as meat juices, nitrates, nitrites, and high concentrations of salt. As described above, cellulase was added at a rate of 1 ml cellulase (2%; 25 FPU/ml) per 50 ml reaction volume inoculated with a 5.0 ml bacterial inoculum of 6 g cells (dry weight)/L. Lactic acid production was high, ranging up to 90% lactic acid per gram spent cellulose casing using L. plantarum in a 26-h incubation at 37° C. Using L. delbrueckii, lactic acid conversion ranged up to 80% lactic acid per gram spent cellulose casing after 36-40 hours at 37° C.

Simultaneous Saccharification and Fermentation to Produce Ethanol

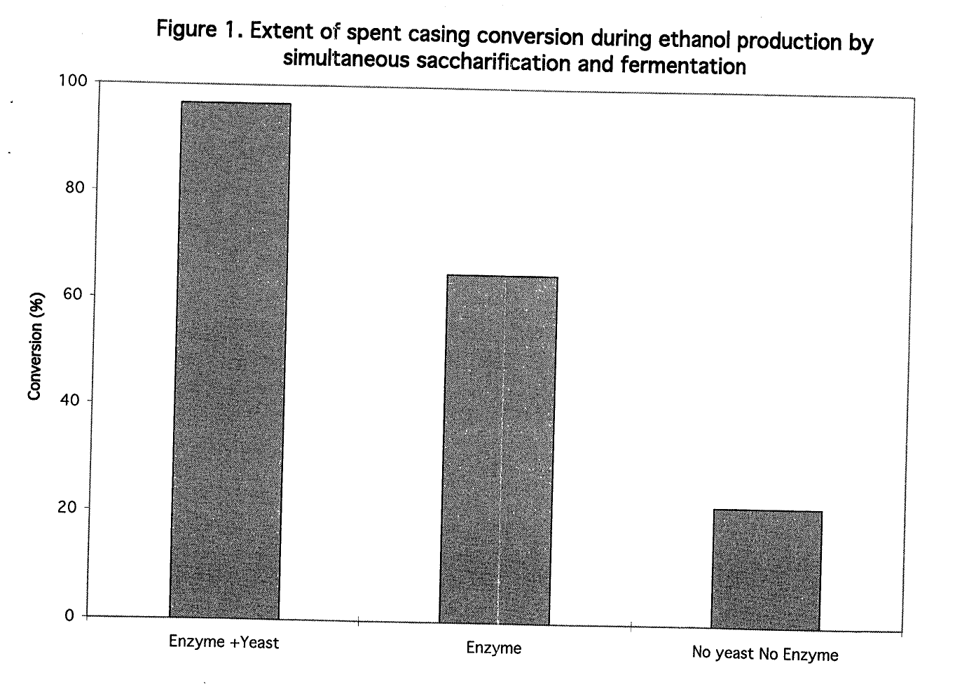

Fermentation medium containing 10% (w/w) spent sausage casings, 50 filter paper units (FPU) cellulase, pH 4.4, was combined with 5 g/L of fresh cell inoculum of Kluveromyces marxianus containing nutrient supplements (1.7 g/L filter-sterilized yeast nitrogen base; 2.27 g/L urea; and 6.56 g/L peptone). The cellulose sausage casings were steam-sterilized, whereas all other fermentation components were added aseptically. Fermentation was conducted in an 125-ml flask with shaking at 100 rpm for 3-5 days at 45° C. The culture filtrate was cleared by centrifugation and the supernatant was analyzed by high performance liquid chromatography for ethanol, sugar, and organic acid content. FIG. 1 shows a comparison of percent conversion of cellulose during ethanol fermentation with yeast and cellulase relative to the percent conversion of cellulose incubated with enzyme only or with cellulose only. With cellulose, greater than 90% of the stoichiometrically achievable quantity of cellulose was converted to ethanol in reactions containing cellulase and yeast.

Preparation of Sausage Casings for Solid Substrate Cultivation of Cellulolytic Fungi

Spent casings are shredded to a size of about 4×0.4 cm and wetted with tap water, to a moisture content of from about 60% to about 80%. The moistened cellulosic material is then placed in a bioreactor and steam-sterilized. Tween 80 (0.1%), which may optionally contain supplemental nutrients such as nutrient nitrogen or mineral salts, are added asceptically and mixed well. Particle size may be selected based on the particular application. The particle size, which affects the porosity of the substrate blend, is an important consideration in SSC because it affects enzyme production efficiency. In small-scale flask culture (<20 L) smaller particle sizes generally result in higher enzyme yields. In larger scale SSC, a variation in particle size (e.g., a mixture of large and small particles, can cause channeling and uneven cell growth, and adversely affect oxygen transfer. Introduction of fibrous material intro the substrate blend generally results in improved porosity.

Following sterilization, the cellulose material is inoculated with a cellulolytic fungal isolate and mixed well. For sporulating fungi, spore suspensions from plate cultures will be used. For non-sporulating organisms, suspensions of blended mycelia will be used. Residual substrate from a previous fermentation may be used as the inoculum.

Solid Substrate Cultivation of Cellulolytic Fungi

Following inoculation of the solid substrate, the fungi are incubated at a temperature of from about 27° C. to about 37° C. for about three days to about eight days.

The majority of carbon and nitrogen used by the fungi during SSC fungal growth are provided by hydrolyzed cellulose and residual meat juices, respectively. Optionally, fungal growth may be enhanced by adding ammonium nitrate or urea (0.2% to 2%), corn steep liquor (1 to 5%), or mineral salts (1 to 10%). A surfactant such as Tween 80 or olive oil may also be used. Nutrients may be reclaimed by recycling spent fermentation broth. Imbalance of carbon and nitrogen sources is detrimental to optimal fungal growth and protein secretion. It may be desirable to periodically adjust the carbon:nitrogen balance by supplementing the SSC with corn steep liquor as a nitrogen source.

Temperature is monitored during growth using embedded thermocouples and regulated by adjusting the humidity and airflow. Bioreactors may be equipped with heat exchangers to remove metabolic heat generated during SSC fungal growth. The fungal cultivation will be conducted at a pH of from about 4.5 to about 6.5. In cultivations conducted at a volume of 20 L or greater, pH of the solid substrate medium will be monitored and adjusted using a suitable acid or base to maintain the pH in the appropriate range.

Moisture content of the SSC may increase due to metabolic activity or decrease due to the flow of air over the substrate. Initial moisture content of SSC cultures will be adjusted to from 60 to 80%. Moisture balances may be determined by measuring relative humidity and airflow through the reactor, and may be adjusted as necessary to maintain suitable moisture content.

Optimal aeration conditions will be determined using maximal pressures in the range of 0 to 20 psi gage and cycles of from 1 to 8 per minute. Homogeneity and porosity of the bed will be assessed by multiple thermocouple readings and by visual inspection. During aeration, air temperature, humidity, and CO₂ concentrations entering and leaving the bioreactor will be monitored. Average airflow rate will be varied to maintain target temperatures. It is assumed that this flow rate will avoid CO₂ concentrations higher than desired. Inlet air will be humidified to 100% relative humidity, except when excess liquid accumulates in the reactor. The rate of metabolic activity will be measured by continuously monitoring the level of CO₂ in the inlet and effluent gas lines using automatic valves and infrared absorption or mass spectrometry. Preferably, the solid substrate is periodically stirred to promote maintenance of the porosity and homogeneity of the substrate mass.

Extraction of Enzymes

At the end of SSC, the fungal residue was extracted at room temperature with tap water using a solid:liquid ratio of 1:3 and the extract was filtered using a Buchner funnel under vacuum. The clear supernatant solution was saved at 4° C. for enzyme assays. Enyzme extraction may not be necessary in other cases because the whole SSC mycelium plus casings will be employed for commercial practice of simultaneous and fermentation of spent casings.

Estimation of Fungal Biomass and Protein

In SSC, fungal mycelia are intimately bound to the solid matrix and cannot be quantitatively separated from the substrate matrix. Therefore, direct measurement of fungal growth is not possible. Various indirect biomass measures including fungal cell constituents, ergosterol, nucleic acids, protein, nitrogen, and chitin CO₂, ATP, and enzyme activity or nutrient consumption have been previously described (Desgranges et al. (Appl. Microbiol. Biotechnol 35:200, 1991; Roche et al. Biotechnol. Adv. 11:67, 1993). Fungal protein will be estimated using a LECO nitrogen analyzer, CO₂ evolution, and microscopic observation. The nitrogen content of residual substrate may be used to estimate protein biomass.

Enzyme Assays

For estimating cellulase and xylanase activity, the biomass residue will be extracted with water using a solid:liquid ratio of 1:3. Cellulases will be assayed by the dinitrosalicylic acid (DNS) method using carboxymethyl cellulose (CMC) as the substrate and by filter paper activity (FPA) (Mandels et al. Biotech. Bioeng. Symp. 6:21, 1976). Xylanase activity will be determined by reducing sugar assays using the DNS method (Miller, Anal. Chem. 31:426, 1959; Patel et al. Appl. Microbiol. Biotechnol. 39:405, 1993). Pectinase assays will be performed according to Sreenath et al. (J. Fd. Sci. 52:230, 1987) and viscosity reduction assays will be conducted as described previously (Sreenath et al. Lebensmittelwissenschaft und Technologie 26:224, 1993).

All publications cited herein are incorporated by reference.

The present invention is not limited to the exemplified embodiments, but is intended to encompass all such modifications and variations as come within the scope of the following claims.

I claim:

1. A method of converting cellulose in cellulose sausage casings to lactic acid comprising the step of:
treating spent cellulose sausage casings with cellulase and a lactic acid producing microorganism under suitable conditions and for a period of time sufficient to allow conversion of at least a portion of the cellulose to lactic acid.

2. The method of claim 1, wherein the microorganism is selected from the group consisting of Lactobacillus species.

3. The method of claim 2, wherein the microorganism is selected from the group consisting of Lactobacillus brevis, Lactobacillus bulgaricus, Lactobacillus delbrueckii, Lactobacillus delbrueckii subsp. lactis, Lactobacillus delbrueckii subsp. bulgaricus, Lactobacillus fermentum, Lactobacillus lactis, Lactobacillus pentosus, Lactobacillus plantarum, and Lactobacillus thermophilus.

4. The method of claim 1, wherein the cellulase is selected from the group consisting of a partially purified cellulase and cellulase contained in or obtained from a solid substrate cultivation of a cellulolytic fungus.

5. The method of claim 4, wherein the cellulase contained in or obtained from a solid substrate cultivation of a cellulolytic fungus is selected from the group consisting of Trichoderma reesei, Rhizopus oryzae, and Aspergillus niger.

6. A method of converting cellulose in cellulose sausage casings to ethanol comprising the step of:
treating spent cellulose sausage casings with cellulase and an ethanol producing microorganism under suitable conditions and for a period of time sufficient to allow conversion of at least a portion of the cellulose to ethanol.

7. The method of claim 6, wherein the cellulase is selected from the group consisting of a partially purified cellulase and cellulase contained in or obtained from a solid substrate cultivation of a cellulolytic fungus.

8. The method of claim 6, wherein the cellulase contained in or obtained from a solid substrate cultivation of a cellulolytic fungus selected from the group consisting of Trichoderma reesei, Rhizopus oryzae, and Aspergillus niger.

9. The method of claim 6, wherein the microorganism is selected from the group consisting of Kluveromyces marxianus and Saccharomyces cerevisiae.

10. A method of producing an enzyme from the solid substrate cultivation of a cellulolytic fungus comprising the steps of:
(a) inoculating spent cellulose sausage casings with a cellulolytic fungus; and
(b) incubating the inoculated casings of step (a) under suitable conditions and for a period of time sufficient to allow the fungus to produce an enzyme selected from the group consisting of cellulase, xylanase, hemicellulase, and pectinase.

11. The method of claim 10 wherein the cellulolytic fungus of step (a) is selected from the group consisting of Trichoderma reesei, Rhizopus oryzae, and Aspergillus niger.

12. A method of producing feed grade protein from the solid substrate cultivation of a cellulolytic fungus comprising the steps of:
(a) inoculating spent cellulose sausage casings with a cellulolytic fungus; and
(b) incubating the inoculated casings of step (a) under suitable conditions and for a period of time sufficient to allow the fungus hydrolyze at least a portion of the cellulose to glucose and to convert at least a portion of the glucose to protein.

13. The method of claim 12 wherein the cellulolytic fungus of step (a) is selected from the group consisting of Trichoderma reesei, Rhizopus oryzae, and Aspergillus niger.
